(19) United States
(12) Patent Application Publication
(10) Pub. No.: US 2021/0356339 A1
(43) Pub. Date: Nov. 18, 2021

(54) FABRIC STRAIN GAUGE, FABRIC PRESSURE GAUGE AND SMART CLOTHING

(71) Applicant: MEDX TECHNOLOGY INC., Taipei City (TW)

(72) Inventors: Chia-Ming Hsu, Taipei (TW); Yi-Yuan Chen, Taipei (TW); Chung-Cheng Wang, Taipei City (TW)

(73) Assignee: MEDX TECHNOLOGY INC., Taipei City (TW)

(21) Appl. No.: 17/321,276

(22) Filed: May 14, 2021

(30) Foreign Application Priority Data
May 14, 2020 (TW) 109116078

(51) Int. Cl. G01L 1/22; G01L 5/10; A41D 1/00

(52) U.S. Cl. CPC G01L 1/2206; A41D 1/002; G01L 5/10

(57) ABSTRACT

A fabric strain gauge includes a knitted fabric and a plurality of conductive measuring fibers, a first high conductivity fiber, and a second high conductivity fiber. The conductive measuring fibers are threaded with the knitted fabric, the first high conductivity conductive fiber is threaded with the knitted fabric, and connected to one or more ends of the conductive measuring fibers, and the second high conductivity fiber is threaded with the knitted fabric, and connected to the other ends of at least part of the conductive measuring fibers. In addition, a fabric pressure gauge and a smart clothing are also disclosed herein.

### TECHNICAL FIELD

One or more embodiments of the present invention relate to a strain gauge and a pressure gauge, in particular to a fabric strain gauge and a fabric pressure gauge applied to a smart clothing.

### BACKGROUND

With the booming technology of electronic devices, a combination of electronic devices and sensing components to provide more diverse services closer to life has become a trend. The state of bodily functions is concerned by most people.

However, if a wearable device is required to be used, a common strain gauge or pressure gauge is not suitable for being worn because of its defects such as poor tactility and inconvenient use. These defects limit the application of pressure sensing components in personal use such as pressure measurement in occasions where sports clothing, smart clothing and shoes are in tight contact with a human body.

In addition, some pressure sensing components having certain sizes are not suitable for frequent measurement, or have to yield to the positions of the sensing components and relevant measuring devices. If a user desires to measure physiological parameters, the user has to move to be beside the measuring products to perform operations.

Conveniently and quickly providing the sensing components required by the wearable devices will help the development and progress of the wearable devices, which in turn improves the quality of human life.

### SUMMARY

A summary aims to provide a simplified abstract of this disclosure so that readers may have a basic understanding of the disclosure. The summary is not a complete description of the present invention, and it is not intended to indicate important/key components of the embodiments of the present invention or define the scope of the present invention.

One or more embodiments of the present invention provide a fabric strain gauge and a fabric pressure gauge, which can be conveniently mounted to a wearable device, in particular to a close-fitting clothing, to provide measurement data of a user's body, so that the user may learn a physical state of the body, and a more comfortable wear experience can be provided.

One or more embodiments of the present invention relate to a fabric strain gauge that includes a knitted fabric, a plurality of conductive measuring fibers threaded with the knitted fabric, a first high conductivity fiber, and a second high conductivity fiber. The conductive measuring fibers are threaded with the knitted fabric, the first high conductivity fiber is also threaded with the knitted fabric and connected to one ends of the conductive measuring fibers, and the second high conductivity fiber is threaded with the knitted fabric and connected to the other ends of at least part of the conductive measuring fibers.

In one or more embodiments, the knitted fabric is a single-sided circular knitted fabric.

In one or more embodiments, the conductive measuring fibers include a first conductive measuring fiber, a second conductive measuring fiber, a third conductive measuring fiber, a fourth conductive measuring fiber, a fifth conductive measuring fiber, a sixth conductive measuring fiber, a seventh conductive measuring fiber, and an eighth conductive measuring fiber, which are arranged parallel to each other.

In one or more embodiments, the fabric strain gauge further includes a third high conductivity fiber threaded with the knitted fabric. The second high conductivity fiber is connected to the other ends of the first conductive measuring fiber, the second conductive measuring fiber, the third conductive measuring fiber and the fourth conductive measuring fiber, and the third high conductivity fiber is connected to the other ends of the fifth conductive measuring fiber, the sixth conductive measuring fiber, the seventh conductive measuring fiber and the eighth conductive measuring fiber.

In one or more embodiments, the fabric strain gauge further includes an auxiliary knitted fabric and a plurality of auxiliary conductive measuring fibers. The auxiliary conductive measuring fibers are threaded with the auxiliary knitted fabric, wherein the first high conductivity fiber is threaded with the knitted fabric and the auxiliary knitted fabric and connected to the one ends of the conductive measuring fibers of the knitted fabric and the auxiliary conductive measuring fibers of the auxiliary knitted fabric, and the second high conductivity fiber is threaded with the knitted fabric and the auxiliary knitted fabric and connected to the other ends of the conductive measuring fibers of the knitted fabric and the auxiliary conductive measuring fibers of the auxiliary knitted fabric.

According to one or more embodiments of the present invention, a fabric pressure gauge is disclosed. The fabric pressure gauge includes a knitted fabric, a plurality of high-impedance conductive fibers, a first silver fiber group, a first high conductivity fiber, a second silver fiber group and a second high conductivity fiber.

The high-impedance conductive fibers are longitudinally threaded with the knitted fabric, the first silver fiber group transversely passes through the high-impedance conductive fibers, the first high conductivity fiber is longitudinally connected to the first silver fiber group, the second silver fiber group transversely passes through the high-impedance conductive fibers, and the second high conductivity fiber is longitudinally connected to the second silver fiber group. The silver fibers of the first silver fiber group and the second silver fiber group are arranged in a staggered manner.

In one or more embodiments, the knitted fabric is a single-sided circular knitted fabric.

In one or more embodiments, the fabric pressure gauge further includes another knitted fabric, wherein the first silver fiber group, the first high conductivity fiber, the second silver fiber group and the second high conductivity fiber are arranged on the another knitted fabric.

According to one or more embodiments of the present invention, a smart clothing is disclosed. The smart clothing includes a wearable fabric, the aforementioned fabric strain gauge or the fabric pressure gauge fixed to the wearable fabric, and a magnetic button group.

In one or more embodiments, the smart clothing further includes a control device detachably fixed to the magnetic button group, and the control device includes a body, a first connection terminal, a second connection terminal and a third connection terminal. The first connection terminal is arranged on the body and electrically connected to a first magnetic button and is attractable to the first magnetic button, the second connection terminal is arranged on the body and electrically connected to a second magnetic button and is attractable to the second magnetic button, and the third connection terminal is arranged on the body and electrically connected to a third magnetic button and is attractable to the third magnetic button.

Therefore, the fabric strain gauge and the pressure gauge may be conveniently and comfortably mounted on the user's clothing, especially the close-fitting clothing, may measure the user's physical information such as physiological response, and may be softly hidden in the close-fitting clothing to reduce the user's discomfort during use so as to measure the user's physiological data more accurately and effectively assist the user to exercise, thereby improving the user's life quality.

### BRIEF DESCRIPTION OF DRAWINGS

In order to make the above and other objectives, features, advantages and embodiments of the present disclosure more apparent and easier to understand, the present disclosure will be described in combination with the drawings:

FIG. 1 is a schematic diagram of a fabric strain gauge according to one or more embodiments of the present invention.

FIG. 2 is a schematic diagram of a fabric strain gauge according to one or more embodiments of the present invention.

FIG. 3 is a schematic diagram of a fabric pressure gauge according to one or more embodiments of the present invention.

FIG. 4 is a schematic diagram of a fabric pressure gauge according to one or more embodiments of the present invention.

FIG. 5 is a schematic diagram of a smart clothing according to one or more embodiments of the present invention.

FIG. 6 is a schematic diagram of a control device of a smart clothing according to one or more embodiments of the present invention.

### DETAILED DESCRIPTION

Embodiments are described in detail below with reference to the accompanying drawings, but the embodiments are not intended to limit the scope of the present disclosure. The description of structural operations is not intended to limit the execution order thereof. Any device recombined by components and having the same effects falls into the scope of the present disclosure. In addition, the drawings are for the purpose of illustration only, and are not drawn to scale. In order to facilitate understanding, the same or similar components will be described with the same reference numerals in the following description.

In addition, the terms used in the description and the claims have general meanings for each of the terms used in this art, in the content disclosed herein and in special content, unless otherwise specified. Some terms used to describe the present disclosure will be discussed below or elsewhere to provide additional guidance about the description of the present disclosure for those skilled in the art.

In the embodiments and the claims, the articles “a” and “the” can be referred to as a single or multiple, unless otherwise defined. The numbers used in steps are only used to indicate the steps for explanation, not to limit the order or implementation.

Moreover, the terms “include”, “comprise”, “have”, “contain” and the like used herein are open terms, and mean to include but not limited to.

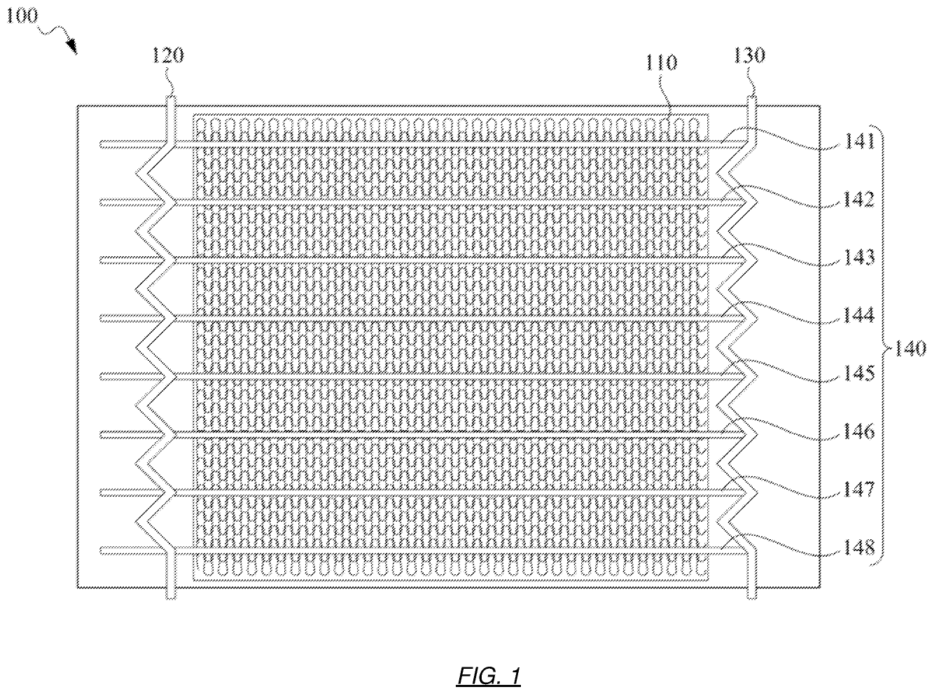

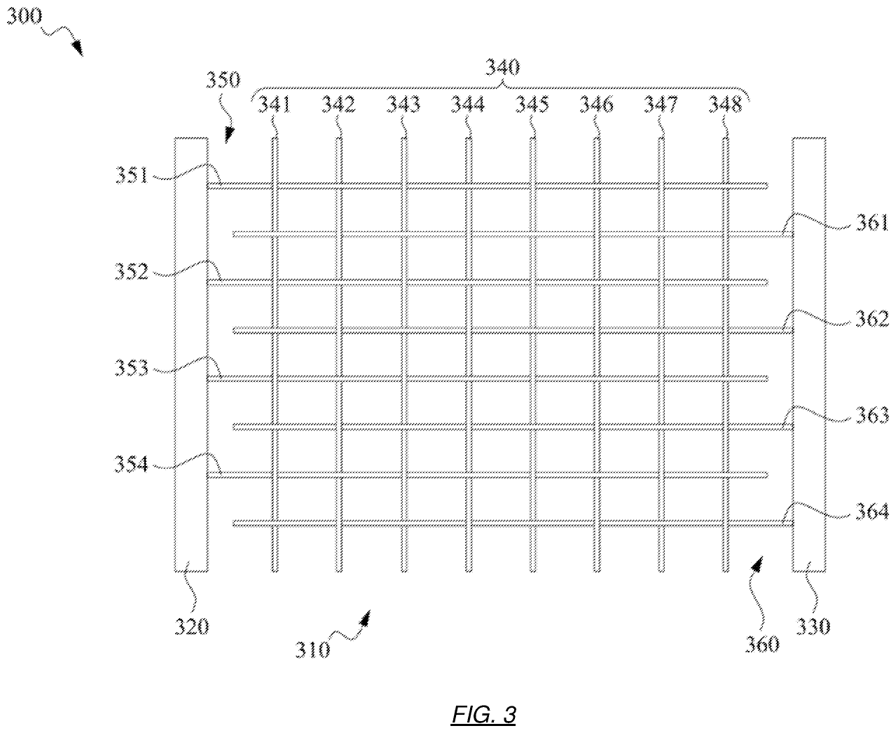

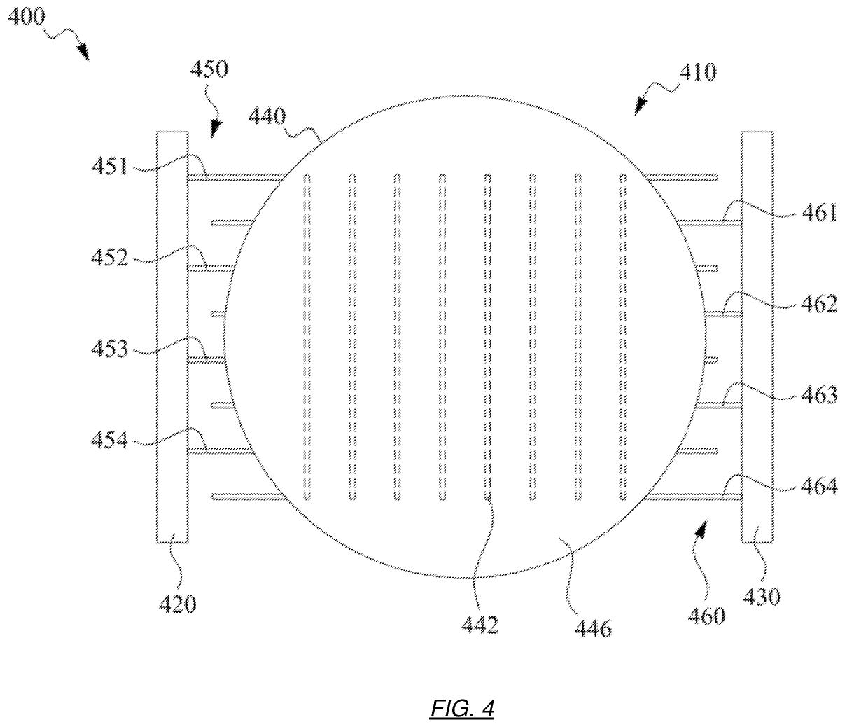

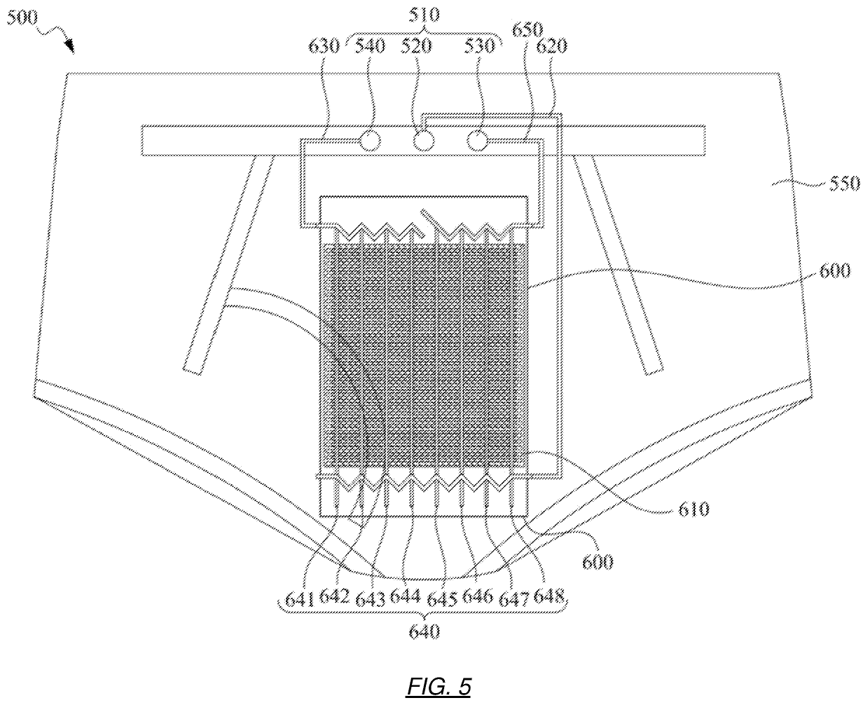

Referring to FIGS. 1 to 5, FIGS. 1 and 2 are respectively schematic diagrams of fabric strain gauges, FIGS. 3 and 4 are respectively schematic diagrams of fabric pressure gauges, and FIG. 5 is a schematic diagram of a smart clothing.

As shown in FIG. 1, a fabric strain gauge 100 includes a knitted fabric 110, a plurality of conductive measuring fibers, for example, a conductive measuring fiber group 140 composed of a first conductive measuring fiber 141, a second conductive measuring fiber 142, a third conductive measuring fiber 143, a fourth conductive measuring fiber 144, a fifth conductive measuring fiber 145, a sixth conductive measuring fiber 146, a seventh conductive measuring fiber 147 and an eighth conductive measuring fiber 148 as shown in the figure, a first high conductivity fiber 120, and a second high conductivity fiber 130. The conductive measuring fiber group 140 is threaded with the knitted fabric 110.

The first high conductivity fiber 120 is threaded with the knitted fabric 110 and connected to one ends of the conductive measuring fibers, for example, left ends of the conductive measuring fibers as shown in the figure. The second high conductivity fiber 130 is threaded with the knitted fabric 110 and connected to the other ends of at least part of the conductive measuring fibers, for example, right ends of the conductive measuring fibers as shown in the figure. The electrical conductivity of the high conductivity fiber is more than 30×10⁶ S·m⁻¹, for example, between 30×10⁶ S·m⁻¹ and 63.01×10⁶ S·m⁻¹.

The conductive measuring fiber changes its impedance value with the deformation of the knitted fabric 110. For example, when the knitted fabric 110 is not stressed, the knitted fabric 110 has little deformation and the impedance value is small. When the knitted fabric 110 bears left and right tensions, the knitted fabric 110 has large deformation, and the impedance value changes with the deformation, for example, if the deformation is larger, the impedance value is larger. Therefore, the fabric strain gauge 100 may effectively measure the deformation of the knitted fabric 110, or measure the tension borne by the knitted fabric 110.

In one or more embodiments, the knitted fabric 110 may be a single-sided circular knitted fabric, and the conductive measuring fibers are threaded with the knitted fabric 110, or the conductive measuring fibers substitute some fibers of the knitted fabric 110 to form a knitted fabric with conductive measuring fibers, which does not depart from the spirit and scope of the present invention.

In one or more embodiments, the fabric strain gauge 100 may employ a double-layer knitted fabric and conductive measuring fibers arranged in parallel, and then the first high conductivity fiber 120 and the second high conductivity fiber 130 are electrically connected to two ends of the conductive measuring fibers on the double-layer knitted fabric.

In one or more embodiments, the fabric strain gauge 100 further includes an auxiliary knitted fabric and a plurality of auxiliary conductive measuring fibers. The auxiliary conductive measuring fibers are threaded with the auxiliary knitted fabric, the first high conductivity fiber 120 is threaded with the knitted fabric 110 and the auxiliary knitted fabric and connected to one ends of the conductive measuring fibers of the knitted fabric 110 and the auxiliary conductive measuring fibers of the auxiliary knitted fabric, and the second high conductivity fiber 130 is threaded with the knitted fabric 110 and the auxiliary knitted fabric and connected to the other ends of the conductive measuring fibers of the knitted fabric 110 and the auxiliary conductive measuring fibers of the auxiliary knitted fabric. The auxiliary knitted fabric and the plurality of auxiliary conductive measuring fibers have the same or similar shapes as the knitted fabric 110 and the conductive measuring fibers on the knitted fabric 110, so that the first high conductivity fiber 120 and the second high conductivity fiber 130 may be connected to the conductive measuring fibers on the double-layer knitted fabric, thereby improving the accuracy and durability of the fabric strain gauge 100 and increasing the measurement range of the fabric strain gauge 100.

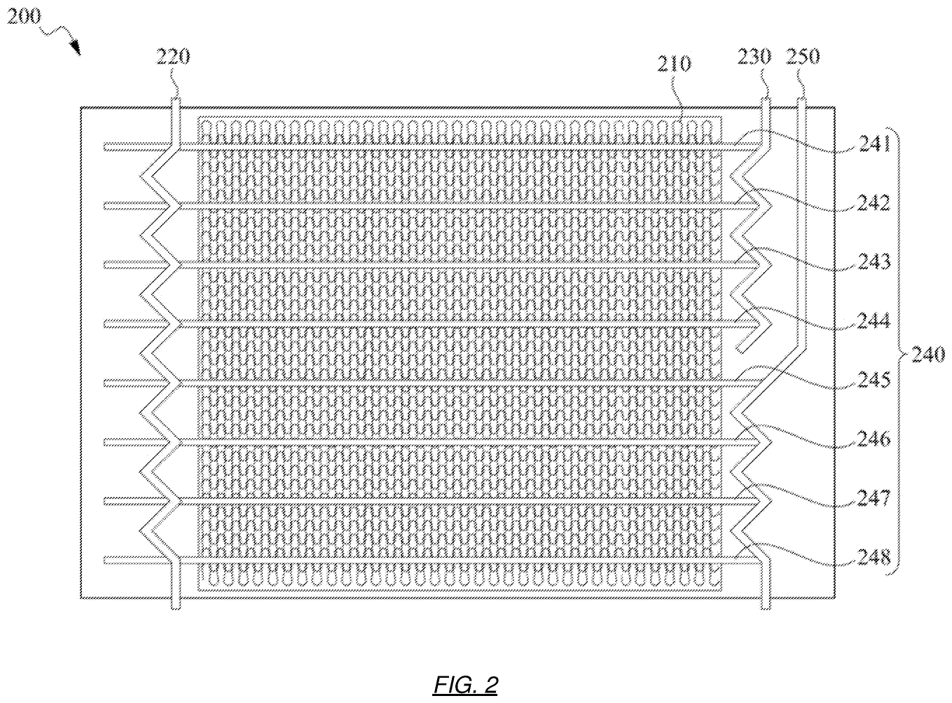

Referring to FIG. 2, a multi-section fabric strain gauge 200 includes a knitted fabric 210, a plurality of conductive measuring fibers, for example, a conductive measuring fiber group 240 composed of a first conductive measuring fiber 241, a second conductive measuring fiber 242, a third conductive measuring fiber 243, a fourth conductive measuring fiber 244, a fifth conductive measuring fiber 245, a sixth conductive measuring fiber 246, a seventh conductive measuring fiber 247 and an eighth conductive measuring fiber 248 as shown in the figure, a first high conductivity fiber 220, a second high conductivity fiber 230, and a third high conductivity fiber 250.

The conductive measuring fiber group 240 is threaded with the knitted fabric 210. The first high conductivity fiber 220 is threaded with the knitted fabric 210 and connected to one ends of the conductive measuring fibers, for example, left ends of the conductive measuring fibers as shown in the figure. The second high conductivity fiber 230 is threaded with the knitted fabric 210 and connected to the other ends of at least part of the conductive measuring fibers, for example, right ends of the conductive measuring fibers as shown in the figure. The third high conductivity fiber 250 is also threaded with the knitted fabric 210 and connected to the other ends of the other part of the conductive measuring fibers, for example, right ends of the conductive measuring fibers as shown in the figure.

In one or more embodiments, the second high conductivity fiber 230 is connected to the right ends of the first conductive measuring fiber 241, the second conductive measuring fiber 242, the third conductive measuring fiber 243 and the fourth conductivity of fiber 244. The third high conductivity fiber 250 is connected to the right ends of the fifth conductive measuring fiber 245, the sixth conductive measuring fiber 246, the seventh conductive measuring fiber 247 and the eighth conductive measuring fiber 248.

Therefore, the fabric strain gauge 200 may measure, section by section, the deformation of the knitted fabric 210 and the tension borne by the knitted fabric 210, thereby further improving the measurement precision and direction or position of the fabric strain gauge 200.

In one or more embodiments, the fabric strain gauge 200 may also have a multi-layer structure, or a measuring range of more sections, which does not depart from the spirit and scope of the present invention.

In one or more embodiments, the conductive measuring fibers of the fabric strain gauge 200 are arranged parallel to each other. The electrical conductivity of the high conductivity fiber is more than 30×10⁶ S·m⁻¹, for example, between 30×10⁶ S·m⁻¹ and 63.01×10⁶ S·m⁻¹.

Referring to FIG. 3, a fabric pressure gauge 300 is disclosed, including a knitted fabric 310, a plurality of high-impedance conductive fibers, for example, a high-impedance conductive fiber group 340 composed of a first high-impedance conductive fiber 341, a second high-impedance conductive fiber 342, a third high-impedance conductive fiber 343, a fourth high-impedance conductive fiber 344, a fifth high-impedance conductive fiber 345, a sixth high-impedance conductive fiber 346, a seventh high-impedance conductive fiber 347 and an eighth high-impedance conductive fibers 348, a first silver fiber group 350, a first high conductivity fiber 320, a second silver fiber group 360, and a second high conductivity fiber 330.

As shown in the figure, the high-impedance conductive fiber group 340 is longitudinally threaded with the knitted fabric 310, the first silver fiber group 350 transversely passes through the high-impedance conductive fibers of the high-impedance conductive fiber group 340, the first high conductivity fiber 320 is longitudinally connected to the first silver fiber group 350, and the second silver fiber group 360 also transversely passes through the high-impedance conductive fibers of the high-impedance conductive fiber group 340. The second high conductivity fiber 330 is longitudinally connected to the second silver fiber group 360. The silver fibers of the first silver fiber group 350 and the second silver fiber group 360 are arranged in a staggered manner.

In one or more embodiments, the first silver fiber group 350 includes a first silver fiber 351, a second silver fiber 352, a third silver fiber 353, and a fourth silver fiber 354. The second silver fiber group 360 includes a first silver fiber 361, a second silver fiber 362, a third silver fiber 363, and a fourth silver fiber 364.

The first silver fiber 361 of the second silver fiber group 360 is interposed between the first silver fiber 351 and the second silver fiber 352 of the first silver fiber group 350, and is connected to the second high conductivity fiber 330 and the first high conductivity fiber 320 respectively.

A resistance value is changed by the contact of the high-impedance conductive fibers with the first silver fiber group 350 and the second silver fiber group 360, and voltage and current signals are transmitted by the first high conductivity fiber 320 and the second high conductivity fiber 330 to a control device to obtain a pressure value borne by the fabric pressure gauge 300. Generally, when the fabric pressure gauge 300 bears a small pressure, the resistance value of the overall fabric pressure gauge 300 is high; when the fabric pressure gauge 300 bears a large pressure, the resistance value of the overall fabric pressure gauge 300 is reduced with the increase in contact of the high-impedance conductive fibers with the silver fibers; therefore, the pressure value borne by the fabric pressure gauge 300 can be effectively measured.

In one or more embodiments, the knitted fabric 310 of the fabric pressure gauge 300 may also be a single-sided circular knitted fabric.

Referring to FIG. 4, a fabric pressure gauge 400 is disclosed, which includes a knitted fabric 446 and a plurality of high-impedance conductive fibers 442 formed thereon to form a yarn conductive fabric 440 of high-impedance conductive fibers. Further, the fabric pressure gauge 400 includes another knitted fabric 410, and a first silver fiber group 450, a first high conductivity fiber 420, a second silver fiber group 460 and a second high conductivity fiber 430 are formed on the another knitted fabric 410. Then, the knitted fabric 446 is stitched or bonded to the another knitted fabric 410, so that when the fabric pressure gauge 400 is stressed, the resistance value is changed by the contact of the high-impedance conductive fibers 442 with the first silver fiber group 450 and the second silver fiber group 460, and voltage and current signals are transmitted by the first high conductivity fiber 420 and the second high conductivity fiber 430 to a control device to obtain a pressure value borne by the fabric pressure gauge 400. Generally, when the fabric pressure gauge 400 bears a small pressure, the resistance value of the overall fabric pressure gauge 400 is high; when the fabric pressure gauge 400 bears a large pressure, the resistance value of the overall fabric pressure gauge 400 is reduced with the increase in contact of the high-impedance conductive fibers with the silver fibers; therefore, the pressure value borne by the fabric pressure gauge 400 may be effectively measured. The first silver fiber group 450 includes a first silver fiber 451, a second silver fiber 452, a third silver fiber 453, and a fourth silver fiber 454. The second silver fiber group 460 includes a first silver fiber 461, a second silver fiber 462, a third silver fiber 463, and a fourth silver fiber 464. The silver fibers of the first silver fiber group 450 are interlaced with the silver fibers of the second silver fiber group 460 in opposite directions, that is, the first silver fiber 461 is arranged between the first silver fiber 451 and the second silver fiber 452 in a staggered manner.

In one or more embodiments, the conductive measuring fibers, the high conductivity fibers and the high-impedance conductive fibers may be woven from metal fibers, such as silver fibers, copper fibers, gold fibers or aluminum fibers, or carbon black fibers, conductive metal compound fibers, or conductive polymer fibers, to provide the required conductivity and impedance, and the required impedance and size may be changed through a wrapping process.

In one or more embodiments, the impedance value of the high-impedance conductive fiber is greater than the impedance value of the high conductivity fiber, for example, when an aluminum metal is used as the high conductivity fiber, its impedance value is 2.82×10⁻⁸ Ωm. At this time, the impedance value of the high-impedance conductive fiber is more than 2.82×10⁻⁸ Ωm, for example, more than 1×10⁻⁷ Ωm or more, which does not depart from the spirit and scope of the present invention.

In one or more embodiments, the knitted fabric 310 of the fabric pressure gauge 400 may also be a single-sided circular knitted fabric.

Referring to FIG. 5, a smart clothing 500 is disclosed, which includes a wearable fabric 550, and the above-mentioned fabric strain gauge or fabric pressure gauge. In FIG. 5, the multi-section fabric strain gauge in FIG. 2 is taken as an example. The wearable fabric 550 is stitched or bonded with a fabric strain gauge 600, and installed with a magnetic button group 510 which is electrically connected to the fabric strain gauge 600.

The fabric strain gauge 600 includes a knitted fabric 610, a plurality of conductive measuring fibers, for example, a conductive measuring fiber group 640 composed of a first conductive measuring fiber 641, a second conductive measuring fiber 642, a third conductive measuring fiber 643, a fourth conductive measuring fiber 644, a fifth conductive measuring fiber 645, a sixth conductive measuring fiber 646, a seventh conductive measuring fiber 647 and an eighth conductive measuring fiber 648 as shown in the figure, a first high conductivity fiber 620, a second high conductivity fiber 630, and a third high conductivity fiber 650.

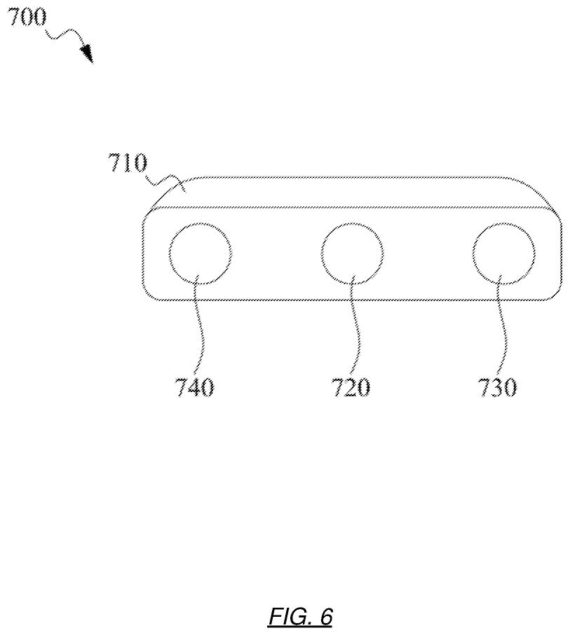

The magnetic button group 510 includes a first magnetic button 520, a second magnetic button 530 and a third magnetic button 540, which are electrically connected to the first high conductivity fiber 620, the third high conductivity fiber 650 and the second high conductivity fiber 630 respectively, to transmit measurement signals to the magnetic button group 510, and then the measurement signals are transmitted to a control device 700 in FIG. 6 by the magnetic button group 510.

Referring to FIG. 6, the control device 700 is detachably fixed to the magnetic button group 510, and the control device 700 includes a body 710 for accommodating relevant components such as a control circuit and a battery module, a first connection terminal 720, a second connection terminal 730, and a third connection terminal 740. The first connection terminal 720 is attracted to the first magnetic button 520 and electrically connected to the first magnetic button 520, the second connection terminal 730 is attracted to the second magnetic button 530 and electrically connected to the second magnetic button 530, and the third connection terminal 740 is attracted to the third magnetic button 540 and electrically connected to the third magnetic button 540.

Therefore, the fabric strain gauge 600 on the wearable fabric 550 of the smart clothing 500 may measure, section by section, the deformation of the knitted fabric 610 and the tension borne by the knitted fabric 610, thereby further improving the measurement precision and direction or position of the fabric strain gauge 600. Therefore, the user can learn actual conditions of the body, especially various physiological data and relevant physiological functions while sleeping at night.

After the control device 700 is connected to a hand-held device, the fabric strain gauge 600 may be controlled by an application in the hand-held device. When an exercise mode or a training mode is used, relevant physiological responses may be continuously recorded, for example, the status and time of erection when sleeping may be recorded, and even the angle and extent of erection may be recorded, which improves the user's understanding on physical conditions, and assists in learning the health status of the body for timely sports or medical treatment, thereby improving life quality.

In summary, the fabric strain gauge and the pressure gauge may be conveniently and comfortably mounted on the user's clothing, especially the close-fitting clothing, may measure the user's physical information such as physiological responses, and may be softly hidden in the close-fitting clothing to reduce the user's discomfort during use so as to measure the user's physiological data more accurately and effectively assist the user to exercise, thereby improving the user's life quality.

Although the present disclosure has been disclosed in the above embodiments, the present disclosure is not limited thereto. Any one skilled in the art can make various changes and modifications without departing from the spirit and scope of the present disclosure. The scope of the appended claims shall prevail over the scope of the present disclosure.

### DESCRIPTION OF REFERENCE NUMERALS

100 Fabric strain gauge
110 Knitted fabric
120 First high conductivity fiber
130 Second high conductivity fiber
140 Conductive measuring fiber group
141 First conductive measuring fiber
142 Second conductive measuring fiber
143 Third conductive measuring fiber
144 Fourth conductive measuring fiber
145 Fifth conductive measuring fiber
146 Sixth conductive measuring fiber
147 Seventh conductive measuring fiber
148 Eighth conductive measuring fiber
200 Fabric strain gauge
210 Knitted fabric
220 First high conductivity fiber
230 Second high conductivity fiber
240 Conductive measuring fiber group
241 First conductive measuring fiber
242 Second conductive measuring fiber
243 Third conductive measuring fiber
244 Fourth conductive measuring fiber
245 Fifth conductive measuring fiber
246 Sixth conductive measuring fiber
247 Seventh conductive measuring fiber
248 Eighth conductive measuring fiber
250 Third high conductivity fiber
300 Fabric pressure gauge
310 Knitted fabric
320 First high conductivity fiber
330 Second high conductivity fiber
340 High-impedance conductive fiber group
341 First high-impedance conductive fiber
342 Second high-impedance conductive fiber
343 Third high-impedance conductive fiber
344 Fourth high-impedance conductive fiber
345 Fifth high-impedance conductive fiber
346 Sixth high-impedance conductive fiber
347 Seventh high-impedance conductive fiber
348 Eight high-impedance conductive fiber
350 First silver fiber group
351 First silver fiber
352 Second silver fiber
353 Third silver fiber
354 Fourth silver fiber
360 Second silver fiber group
361 First silver fiber
362 Second silver fiber
363 Third silver fiber
364 Fourth silver fiber
400 Fabric pressure gauge
410 Knitted fabric
420 First high conductivity fiber
430 Second high conductivity fiber
440 Yarn conductive fabric of high-impedance conductive fibers
442 High-impedance conductive fiber
446 Knitted fabric
450 First silver fiber group
451 First silver fiber
452 Second silver fiber
453 Third silver fiber
454 Fourth silver fiber
460 Second silver fiber group
461 First silver fiber
462 Second silver fiber
463 Third silver fiber
464 Fourth silver fiber
500 Smart clothing
510 Magnetic button group
520 First magnetic button
530 Second magnetic button
540 Third magnetic button
550 Wearable fabric
600 Fabric strain gauge
610 Knitted fabric
620 First high conductivity fiber
630 Second high conductivity fiber
640 Conductive measuring fiber group
641 First conductive measuring fiber
642 Second conductive measuring fiber
643 Third conductive measuring fiber
644 Fourth conductive measuring fiber
645 Fifth conductive measuring fiber
646 Sixth conductive measuring fiber
647 Seventh conductive measuring fiber
648 Eighth conductive measuring fiber
650 Third high conductivity fiber
700 Control device
710 Body
720 First connection terminal
730 Second connection terminal
740 Third connection terminal

1. A fabric strain gauge, comprising:
a knitted fabric;
a plurality of conductive measuring fibers threaded with the knitted fabric;
a first high conductivity fiber threaded with the knitted fabric and connected to one or more ends of the conductive measuring fibers; and
a second high conductivity fiber threaded with the knitted fabric and connected to other ends of at least part of the conductive measuring fibers.

2. The fabric strain gauge of claim 1, wherein the knitted fabric is a single-sided circular knitted fabric.

3. The fabric strain gauge of claim 1, wherein the conductive measuring fibers comprise a first conductive measuring fiber, a second conductive measuring fiber, a third conductive measuring fiber, a fourth conductive measuring fiber, a fifth conductive measuring fiber, a sixth conductive measuring fiber, a seventh conductive measuring fiber, and an eighth conductive measuring fiber arranged parallel to each other.

4. The fabric strain gauge of claim 3, further comprising a third high conductivity fiber threaded with the knitted fabric, wherein the second high conductivity fiber is connected to the other ends of the first conductive measuring fiber, the second conductive measuring fiber, the third conductive measuring fiber and the fourth conductive measuring fiber, and the third high conductivity fiber is connected to the other ends of the fifth conductive measuring fiber, the sixth conductive measuring fiber, the seventh conductive measuring fiber and the eighth conductive measuring fiber.

5. The fabric strain gauge of claim 1, further comprising:
an auxiliary knitted fabric; and
a plurality of auxiliary conductive measuring fibers threaded with the auxiliary knitted fabric, wherein the first high conductivity fiber is threaded with the knitted fabric and the auxiliary knitted fabric and connected to one ends of the conductive measuring fibers of the knitted fabric and the auxiliary conductive measuring fibers of the auxiliary knitted fabric, and the second high conductivity fiber is threaded with the knitted fabric and the auxiliary knitted fabric and connected to the other ends of the conductive measuring fibers of the knitted fabric and the auxiliary conductive measuring fibers of the auxiliary knitted fabric.

6. A fabric pressure gauge, comprising:
a knitted fabric;
a plurality of high-impedance conductive fibers that is longitudinally threaded with the knitted fabric;
a first silver fiber group that transversely passes through the high-impedance conductive fibers;
a first high conductivity fiber that is longitudinally connected to the first silver fiber group;
a second silver fiber group that transversely passes through the high-impedance conductive fibers; and
a second high conductivity fiber that is longitudinally connected to the second silver fiber group, wherein silver fibers of the first silver fiber group and the second silver fiber group are arranged in a staggered manner.

7. The fabric pressure gauge of claim 6, wherein the knitted fabric is a single-sided circular knitted fabric.

8. The fabric pressure gauge of claim 7, further comprising another knitted fabric, wherein the first silver fiber group, the first high conductivity fiber, the second silver fiber group and the second high conductivity fiber are arranged on the another knitted fabric.

9. A smart clothing, comprising:
a wearable fabric,
the fabric strain gauge of claim 1, fixed to the wearable fabric; and
a magnetic button group arranged on the wearable fabric and electrically connected to the fabric strain gauge.

10. The smart clothing of claim 9, further comprising a control device detachably fixed to the magnetic button group, wherein the magnetic button group comprises a first magnetic button and a second magnetic button, and the control device comprises:
a body;
a first connection terminal arranged on the body, electrically connected to the first magnetic button, and magnetically attracted to the first magnetic button; and
a second connection terminal arranged on the body, electrically connected to the second magnetic button, and magnetically attracted to the second magnetic button.

11. A smart clothing, comprising:
a wearable fabric, the fabric pressure gauge of claim 6, fixed to the wearable fabric; and
a magnetic button group arranged on the wearable fabric and electrically connected to the fabric pressure gauge;
a control device detachably fixed to the magnetic button group, wherein the magnetic button group comprises a first magnetic button and a second magnetic button, and the control device comprises:
a body;
a first connection terminal arranged on the body, electrically connected to the first magnetic button, and magnetically attracted to the first magnetic button; and
a second connection terminal arranged on the body, electrically connected to the second magnetic button, and magnetically attracted to the second magnetic button.

12. A smart clothing, comprising:
a wearable fabric,
the fabric strain gauge of claim 2, fixed to the wearable fabric; and
a magnetic button group arranged on the wearable fabric and electrically connected to the fabric strain gauge.

13. A smart clothing, comprising:
a wearable fabric,
the fabric strain gauge of claim 3, fixed to the wearable fabric; and
a magnetic button group arranged on the wearable fabric and electrically connected to the fabric strain gauge.

14. A smart clothing, comprising:
a wearable fabric,
the fabric strain gauge of claim 4, fixed to the wearable fabric; and
a magnetic button group arranged on the wearable fabric and electrically connected to the fabric strain gauge.

15. A smart clothing, comprising:
a wearable fabric,
the fabric strain gauge of claim 5, fixed to the wearable fabric; and
a magnetic button group arranged on the wearable fabric and electrically connected to the fabric strain gauge.

16. A smart clothing, comprising:
a wearable fabric,
the fabric pressure gauge of claim 7, fixed to the wearable fabric; and
a magnetic button group arranged on the wearable fabric and electrically connected to the fabric pressure gauge;
a control device detachably fixed to the magnetic button group, wherein the magnetic button group comprises a first magnetic button and a second magnetic button, and the control device comprises:
a body;
a first connection terminal arranged on the body, electrically connected to the first magnetic button, and magnetically attracted to the first magnetic button; and
a second connection terminal arranged on the body, electrically connected to the second magnetic button, and magnetically attracted to the second magnetic button.

17. A smart clothing, comprising:
a wearable fabric,
the fabric pressure gauge of claim 8, fixed to the wearable fabric; and
a magnetic button group arranged on the wearable fabric and electrically connected to the fabric pressure gauge;
a control device detachably fixed to the magnetic button group, wherein the magnetic button group comprises a first magnetic button and a second magnetic button, and the control device comprises:
a body;
a first connection terminal arranged on the body, electrically connected to the first magnetic button, and magnetically attracted to the first magnetic button; and
a second connection terminal arranged on the body, electrically connected to the second magnetic button, and magnetically attracted to the second magnetic button.

18. The smart clothing of claim 11, wherein the control device obtains a pressure value borne by the fabric pressure gauge.

19. The smart clothing of claim 18, wherein the pressure value is included in voltage and current signals transmitted by the first high conductivity fiber and the second high conductivity fiber to the control device.

20. The smart clothing of claim 18, wherein a resistance of the fabric pressure gauge is determined based on the pressure value.
